(19) United States
(12) Patent Application Publication
(10) Pub. No.: US 2011/0197668 A1
(43) Pub. Date: Aug. 18, 2011

(54) APPARATUS FOR ADJUSTING FOOT STRUCTURES, FOR DESIGN OF A FOOT ORTHOTIC, AND METHODS OF USE

(75) Inventors: J. Kevin Miller, Jackson, AL (US); Neal J. Beidleman, Aspen, CO (US); Neville A. Bonwit, Newark, CA (US); Luke Clauson, Redwood City, CA (US); Kenneth J. den Dulk, Davis, CA (US); Gregg E. Freebury, Lafayette, CO (US); Matthew B. Newell, Redwood City, CA (US)

(73) Assignee: Tensegrity Technologies, Inc., Mill Valley, CA (US)

(21) Appl. No.: 13/025,026

(22) Filed: Feb. 10, 2011

Related U.S. Application Data
(60) Provisional application No. 61/303,554, filed on Feb. 11, 2010.

(51) Int. Cl. A61B 5/103

(52) U.S. Cl. 73/172

(57) ABSTRACT

An apparatus comprised of a plurality of engagement structures independently movable along a longitudinal axis to initially engage a mid-foot region of a foot is described. A center structure or a first set of engagement structures engage the foot in a mid-foot region, and one or more peripheral engagement structures engage the plantar surface in regions surrounding the mid-foot region independent from the structure(s) engaging the mid-foot region. Positional information about the engagement structures is obtained, and a surface map from the positional information is constructed, to determine a profile or contour for an orthotic device in which the foot is in a restored bone state.

### CROSS-REFERENCE TO RELATED APPLICATIONS

This application claims the benefit of U.S. provisional application Ser. No. 61/303,554, filed Feb. 11, 2010, incorporated by reference herein.

### TECHNICAL FIELD

The subject matter described herein relates to an apparatus for adjusting a structure, such as a bone, in a foot, and for design of a foot orthotic with the foot structures in an adjusted state. More particularly, the subject matter is directed to an apparatus that adjusts structures, e.g., bones and soft tissue, of a foot to a desirable corrective position or alignment, and provides a graphic image of a surface contour of a corrective orthotic device that maintains the desirable corrective position or alignment of the foot structures.

### BACKGROUND

There are two basic types of custom foot orthoses made today, accommodative orthoses and functional orthoses. An accommodative orthosis is typically made from a soft or flexible material that cushions and “accommodates” any deformity of the foot. This cushioning also results in some dissipation of the forces required for efficient gait that ordinarily would be transmitted up the kinetic chain. Accommodative orthosis, which are typically made of soft or cushioning materials, are unable to control foot mechanics.

A functional foot orthosis is one that controls joint movements and/or foot position. Functional foot orthoses are typically rigid, and clinicians utilize them to hold the foot in a position deemed corrective or therapeutic. This approach is problematic because the foot must be allowed to remain mobile to continually adapt to the ground in order to operate efficiently.

Foot orthotics are typically designed based on an exact contour or image of the plantar surface of a patient's foot, and there are a variety of instruments and systems for obtaining the exact contour, including mechanical approaches, such as impression molds using plaster, sand, or foam, and electronic approaches, such as electro-mechanical and electro-optical devices. The available approaches generally provide a mechanical or digital representation of the sensed contour or topography of the foot, absent any individualized, restorative adjustment of foot structures (e.g., bones or soft tissues). While functional and accommodative orthotics may temporarily decrease foot pain due to restricting pathologic range of motion and in cushioning the foot, they necessarily cause pathologic gait, and this approach will inevitably cause pain in other joints in the foot, leg, pelvis and/or back as they compensate for this abnormal motion. There remains a need for an apparatus that generates an image or contour of a foot's plantar surface when the foot's structures are adjusted to a restored position, from which a foot orthosis can be constructed that corrects and/or restores the alignment and/or positioning of foot structures.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In a first aspect, an apparatus comprising a plurality of engagement structures and one or more biasing members is provided. Each pin is independently movable along a longitudinal axis, and the one or more biasing members is configured to exert a force on one or more pins in the plurality of pins, such that a first set of pins in the plurality of pins moves from a first position to a second position independent of a second set of pins in the plurality of pins.

In one embodiment, the one or more biasing members exerts a force to achieve movement of the one or more pins in the plurality of pins. In another embodiment, the one or more biasing members resists a force applied to one or more pins in the plurality of pins.

In one embodiment, pins in the first set of pins are moved prior to movement of pins in the second set of pins. In another embodiment, pins in the first set of pins are moved by a first force applied by a biasing member in the one or more biasing members to the pins in the first set of pins, the first force different from a second force applied by a biasing member in the one or more biasing members to pins in the second set of pins.

In another embodiment, the one or more biasing members comprise at least two biasing members.

In yet another embodiment, a first biasing member is dedicated to achieve movement of the first set of pins and a second biasing member is dedicated to achieve movement of the second set of pins.

In other embodiments, the one or more biasing members comprise a plurality of biasing members, for example, the plurality comprises 2, 3, 4, 5, 6, 7, 8, 9, 10, 11 or more biasing members. In other embodiments, each biasing member is dedicated for achieving movement of a single pin in the plurality of pins or of a single set of pins in the plurality of pins.

In one embodiment, the one or more biasing members are comprised of a pressurized fluid. In yet another embodiment, a first biasing member exerts a first pressure on pins in the first set of pins to move the pins in the first set of pins along their longitudinal axes, and a second biasing member exerts a second pressure on pins in the second set of pins to move the pins in the first set of pins along their longitudinal axes, where the first pressure is different that the second pressure. In one embodiment, the first pressure is higher than the second pressure.

In still other embodiments, the one or more biasing members is configured for contact with a third set of pins in the plurality of pins such that pins in the third set move along their longitudinal axis independent from pins in the first set of pins or the second set of pins.

In one embodiment, the third set of pins are moved subsequent to movement of pins in the first set of pins, and in another embodiment, the third set of pins are moved by a pressure applied to the third set of pins that is different from a pressure applied to the first set of pins. In another embodiment, the third set of pins are moved at a time and at a pressure different from the first and/or second set(s) of pins. Similarly, in some embodiments, the second set of pins are moved at a pressure different from the first set of pins, at a time different from the first set of pins, or both.

In some embodiments, the first set of pins is within a center region of the plurality of pins and the second set of pins surround the periphery of the center region, or in other words, are in a non-center regions of the plurality of pins.

The plurality of pins collectively define an upper surface and a lower surface, and in one embodiment, the one or more biasing members is a single, movable biasing member that contacts the lower surface of the plurality of pins.

In yet other embodiments, the apparatus further comprises a sensor to determine a position of one or more pins within the plurality of pins.

In another aspect, an apparatus comprised of a plurality of pins and at least one biasing member is provided, wherein the plurality of pins is supported with a frame or housing, and each pin is independently movable along a longitudinal axis. One or more biasing members is disposed within the frame, where the one or more biasing members is configured for contact with one or more pins in the plurality of pins such that a first set of pins in the plurality of pins is moved along the longitudinal axis of each pin in the first set at a pressure and/or at a time different from movement of a second set of pins in the plurality of pins along the longitudinal axis of each pin in the second set.

In one embodiment, the one or more biasing members comprise a plurality of biasing members. In another embodiment, each biasing member in the plurality of biasing members is dedicated for movement of a single pin in the plurality of pins or of a single set of pins in the plurality of pins.

In another embodiment, a first biasing member is dedicated for urging pins in the first set of pins from a first position to a second position (e.g., from an initial position to an engagement position), and a second biasing member is dedicated for urging pins in the second set of pins from a first position to a second position (e.g., from an initial position to an engagement position).

In another embodiment, the one or more biasing members is configured for direct or indirect contact with sets of pins, and in another embodiment, for direct or indirect contact with a third set of pins in the plurality of pins such that pins in the third set move along their longitudinal axis at a pressure or at a time (e.g, subsequent to) different than pins in the first set of pins and/or the second set of pins.

In another embodiment, the biasing member has an upper surface for contact with the lower surface of the plurality of pins, and wherein the upper surface of the biasing member has a pre-selected contour to contact the first set of pins prior to contact with the second set of pins. In a more specific embodiment, the biasing member is rectangular, and the upper surface has a pyramid-like contour with an apex offset from a center point of the rectangle. In another specific embodiment, the biasing member is rectangular, and the upper surface has a terraced contour with an uppermost terrace offset from a center point of the rectangle. In yet another specific embodiment, the biasing member consists of tens sides, and wherein five of the ten sides are on the upper surface. In yet another specific embodiment, the pre-selected contour of the biasing member is a pyramid-like shape with a flat apex.

In another embodiment, the biasing member is composed of a first material having a first density and a second material having a second density. In another embodiment, the biasing member or members is/are composed of a fluid, preferably a gas, that can be pressurized to urge pins from first to second positions. In other embodiments, the biasing member or members is/are a force that act directly or indirectly on one or more pins to effect movement of the pin(s) from first to second positions. Exemplary forces include magnetic force, a pneumatic force or pressure, a pressurized fluid force, gravitational force, a mechanical force and the like.

In another embodiment, the biasing member is composed of a first material having a first durometer and a second material having a second durometer. In a specific embodiment, the first material is a viscoelastic foam. In other specific embodiments, the biasing member is composed of a rubber, an elastomer, a plastic, or a foam.

In yet another embodiment, the apparatus further comprises a locking member to secure one or more pins in the plurality of pins.

In still another embodiment, the apparatus further comprises a sensor to determine a position of one or more pins within the plurality of pins. In a specific embodiment, the apparatus comprises a single sensor that determines the position of each pin in the plurality. In another specific embodiment, the apparatus comprises two or more sensors. In various specific embodiments, the sensor is a non-contact sensor and exemplary non-contact sensors include a laser, such as a one-dimensional laser, a two-dimensional laser, or a three-dimensional laser, and an optical distance scanner. The apparatus can optionally include a reflective surface positioned to reflect a beam from a laser sensor. In yet another specific embodiment, the sensor comprises a plurality of cameras for obtaining images of the plurality of pins from a plurality of angles.

In another embodiment, each pin in the plurality of pins has a diameter between 0.0624 inches to 0.250 inches.

In still another embodiment, the apparatus further comprises a transducer, such as a hall sensor or capacitive sensor, associated with the one or more biasing members.

In yet another embodiment, the apparatus comprises a sensor to determine relative movement of the one or more biasing members.

In still another embodiment, the plurality of pins and the one or more biasing members are capable of producing a force per pin of between about 0.02-4.0 lb-f, more preferably between about 0.02-5 lb-f.

In another aspect, a method for obtaining a restored bone state in a foot and/or for constructing a foot orthotic is provided. The method comprises placing a plantar surface of a foot on an apparatus as described herein, the plantar surface placed on an upper surface defined by the plurality of pins, wherein said plurality of pins are in an initial position. Movement of pins in the plurality of pins is initiated, via the one or more biasing members, such that a first set of pins adjusts one or more bones in the foot to a restored bone state and a second set of pins engages the foot plantar surface with the foot in its restored bone state. A position of each pin in at least the first pin set and the second pin set is determined, to obtain a profile of the foot in its restored bone state, from which an orthotic for the foot can be constructed.

In one embodiment, the method further comprises transferring positional information of each pin to a computer. In another embodiment, a position of each pin in at least the first pin set and the second pin set is determined, to obtain a profile for construction of a foot orthotic or a series of foot orthotics to be worn sequentially.

In yet another embodiment, the first set of pins adjusts one or more bones in the mid-foot region, or localized mid-foot region, of the foot, and a third set of pins in the plurality of pins moves via the one or more biasing members, such that pins in the third set of pins engage the foot plantar surface at a region other than the mid-foot region.

In yet another aspect, a method is provided, wherein the method comprises placing a plantar surface of a foot an apparatus comprising (i) a plurality of pins, wherein each pin in the plurality of pins is independently movable along a longitudinal axis, and (ii) one or more biasing members configured for contact with one or more pins in the plurality of pins, such that a first set of pins in the plurality of pins is moved along the longitudinal axis of each pin in the first set independent of a second set of pins in the plurality of pins. Movement of the one or more biasing members is effected, such that the first set of pins adjusts one or more bones in the foot to an adjusted position; and a position of each pin in at least the first pin set and the second pin set is determined.

In one embodiment, the first set of pins adjusts one or more bones in the foot to a restored bone position. In another embodiment, the first and second sets of pins engage the foot in order to achieve a restored bone position.

In one embodiment, movement of the pins or a biasing member is such that the first set of pins adjusts one or more mid-foot bones to an adjusted position.

In another embodiment, the one or more biasing members is a fluid, and pressurizing the fluid effects movements of one or more pins or sets of pins in the plurality.

In another embodiment, a first biasing member has a first pressure, to achieve movement of the first pin set, and a second biasing member has a second pressure, to achieve movement of the second pin set. The first pressure and the second pressure can be the same or different.

In other embodiments, the position of an end of each pin in at least the first pin set and the second pin set that engages the foot plantar surface are determined, to obtain a positional point of each pin, the positional points collectively defining a surface map.

In one embodiment, the method further comprises after the step of causing, locking the plurality of pins to secure each pin in a final position.

In another embodiment, the step of determining comprises determining by means of the sensor a position of each pin.

In still another embodiment, the method further comprises transferring positional information of each pin to a computer to construct a digital image of the profile.

In another embodiment, the step of determining comprises determining a position of each pin in at least the first pin set and the second pin set to obtain a profile for construction of a series of foot orthotics to be worn sequentially.

In another aspect, a method comprises providing an apparatus comprising (i) a plurality of pins, wherein each pin in the plurality of pins is independently movable along a longitudinal axis, and (ii) one or more biasing members configured for contact with one or more pins in said plurality of pins such that a first set of pins in the plurality of pins is moved along the longitudinal axis of each pin in the first set independent (e.g., prior to or at a different pressure) movement of a second set of pins in said plurality of pins along the longitudinal axis of each pin in the second set of pins; placing a plantar surface of a foot on the plurality of pins; causing movement of the one or more biasing members such that some of all of the pins in the first set of pins adjusts one or more bones in the foot to an adjusted position, and the second set of pins engages the foot plantar surface at positions responsive to the one or more bones in the adjusted position; and after the step of causing, determining a position of each pin in at least the first pin set and the second pin set.

In another embodiment, the first set of plurality of pins contacts a foot plantar surface in a pre-determined or pre-selected force, contour or pattern to produce mid-tarsal movement sufficient to produce tension in a dorsal ligament thereby producing an adjusted position of the foot. Second and subsequent sets of pins in the plurality are contact the foot in its adjusted position, the second and subsequent plurality of pins contacting the foot in its adjusted bone position or restored bone state at a time or force different or the same as the first pin set.

In another aspect, a method is provided, wherein the method comprises engaging a center engagement structure against a localized mid-foot region of a plantar surface of a subject's foot to adjust one or more mid-foot bones into a restored bone state; engaging one or more peripheral engagement structures against a region other than the mid-foot region to contact the plantar surface while maintaining the engagement of the center structure; obtaining positional information of the engagement structures; and based on the positional information, determining a surface map or orthotic profile.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1B are views of an individual's right foot, shown as a top plan view (FIG. 1A) and a side view from the right (FIG. 1B);

FIGS. 1C-1E are a top plan views of a right foot, showing the 1^{st}, 2^{nd}, and 5^{th} rays (FIG. 1C), the mid-foot region and the localized mid-foot region (FIG. 1E).

FIGS. 2A-2B is a perspective view (FIG. 2A) and a side view (FIG. 2B) of an embodiment of the apparatus described herein;

FIGS. 3A-3B are simplified illustrations of an embodiment of the apparatus, where a biasing member provides differential movement of pins in a plurality of pins, where the pins in an initial resting position (FIG. 3A) are urged along their longitudinal axes by a contoured biasing member (FIG. 3B);

FIG. 4 is a illustration of another embodiment of a biasing member having a preselected surface contour to provide differential movement of pins in a pin bed array;

FIG. 5 is a top view of a pin bed showing a center band region of pins;

FIGS. 6A-6B are simplified illustrations of another embodiment of the apparatus, where a biasing member provides differential movement of pins in a plurality of pins, where the pins in an initial resting position (FIG. 6A) are urged along their longitudinal axes upon inflation of a biasing member (FIG. 6B);

FIGS. 7A-7C are simplified illustrations of another embodiment of the apparatus, wherein differential, independent movement of each pin in a plurality of pins is achieved by individual electronics assigned to each pin, where the pins in an initial resting position (FIG. 7A) are urged along their longitudinal axes upon activation or electronic signaling (FIGS. 7B-7C);

FIG. 8 illustrates another embodiment wherein differential, independent movement of pins, or engagement structures, is achieved by a spring associated with each pin;

FIGS. 9A-9C are simplified illustrations of another embodiment of the apparatus, wherein differential, independent movement of sets of pins is achieved pneumatically via pressurized fluid as the biasing member, where the pins in an initial resting position (FIG. 9A) are moved in sets in response to pressure in a zone in fluid communication with each set (FIGS. 9B-9C);

FIGS. 10A-10B illustrate in top view (FIG. 10A) and in cross-sectional view (FIG. 10B) another embodiment of an array of engagement structures that engages a localized mid-foot region of a foot plantar surface to manipulate bones into an adjusted or restored state, with subsequent engagement by peripheral engagement structures of regions surrounding the mid-foot region;

FIG. 11A is a perspective view of another embodiment, comprising a pin bed array and a biasing member associated with each pin or with zones of pins in the array to permit differential, independent movement of the pins or zones of pins along their longitudinal axes;

FIG. 11B is a perspective view of another embodiment comprising a pin bed array and a biasing member that provides differential, independent movement of the pins along their longitudinal axes in accord with the surface contour of the biasing member;

FIGS. 12A-12C illustrate in sequence operation of an exemplary apparatus, where pins are in initial resting positions (FIG. 12A), a zone of pressurized gas acts as a biasing member to effect movement of pins or pin sets in a desired pattern or sequence of movement (FIG. 12B), and the position of each pin or pin set is ascertained (FIG. 12C); and

FIGS. 13A-13C illustrate in sequence operation of another exemplary apparatus, where the pins are in initial resting positions and the biasing member is retracted (FIG. 13A), the biasing member is moved into position to urge the pins upward (FIG. 13B), and the position of each pin is ascertained (FIG. 13C).

### DETAILED DESCRIPTION

### I. Definitions

As used throughout the present disclosure, the technical and scientific terms used in the descriptions herein will have the meanings commonly understood by one of ordinary skill in the art, unless specifically defined otherwise.

As used in this specification and the appended claims, the singular forms “a,” “an,” and “the” include plural referents unless the context clearly indicates otherwise. Thus, for example, reference to a patient's “foot” can include both feet, reference to an “orthotic device” includes a single device as well as two or more of the same or different devices, and reference to a “tarsal bone” refers to a single tarsal bone as well as two or more tarsal bones. The use of “or” should be understood to mean “and/or” unless stated otherwise. Similarly, “comprise,” “comprises,” “comprising” “include,” “includes,” “including,” “has,” “have” and “having” are interchangeable and not intended to be limiting. It is also to be understood that where descriptions of various embodiments use the term “comprising,” those skilled in the art would understand that in some specific instances, an embodiment can be alternatively described using language “consisting essentially of or “consisting of.”

### II. Apparatus

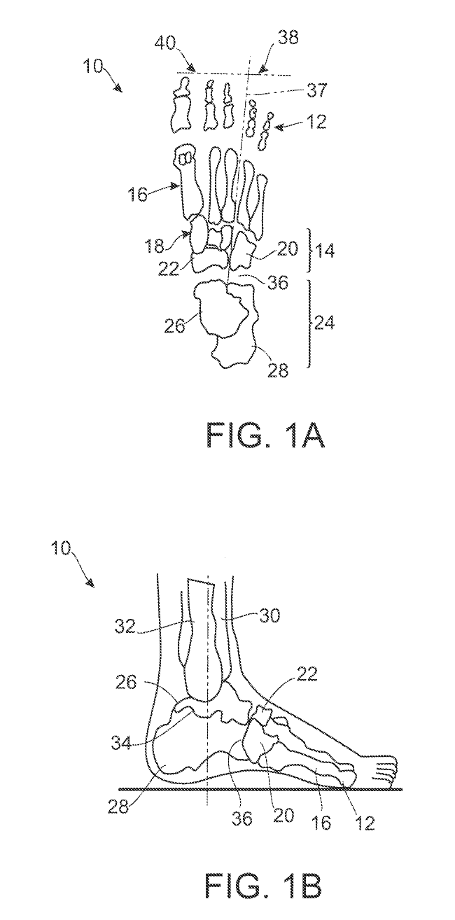

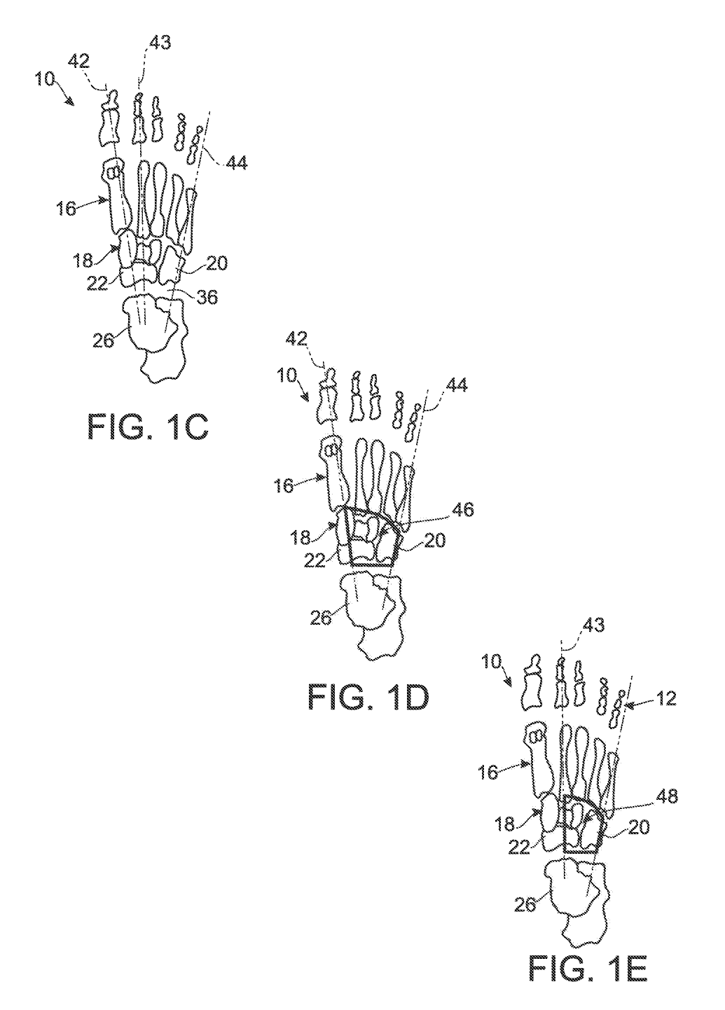

Before discussing the subject apparatus and its methods of use, a brief anatomy of the foot is provided, with reference to FIGS. 1A-1B which depict bones and joints in a right foot and lower leg 10. As seen in FIG. 1A, the phalanges 12 form the toes and connect to the metatarsals 16. Together, the five phalanges and the five metatarsals form the “forefoot”. As a point of reference, the first metatarsal bone is commonly known as the ‘big toe’ and typically bears the most weight in the forefoot and plays a role in propulsion. The “mid-foot” region 14 includes the three cuneiform bones, lateral, middle and medial, generally designated at 18, the cuboid bone 20 and the navicular bone 22. The distal row of the midfoot contains the three cuneiforms and the cuboid and is bounded distally by the metatarsal bones. The proximal row of the midfoot consists of the cuboid and the navicular. The three cuneiforms articulate proximally with the navicular bone. The “rear foot” 24 includes the talus 26 and the calcaneus 28. The calcaneus is the largest tarsal bone, and forms the heel. The talus rests on top of the calcaneus, which interconnects the foot to the tibia 30 and the fibula 32. A subtalar joint 34 constitutes the interface between the talus 26 and the calcaneus 28. A midtarsal joint 36 comprises the interface between cuboid bone 20, navicular bone 25, talus bone 26 and calcaneus bone 28.

The foot is typically divided into two columns. As shown in FIG. 1A, a lateral or lateral load-bearing column is generally designated by identifier 38, which is to the right of dashed line 37. Lateral load-bearing column 38 comprises the calcaneus 28, the cuboid 20 and the fourth and fifth rays of the metatarsals 16 and of the phalanges 12. This represents the outer portion of the foot including the fourth and fifth toes. A medial or medial dynamic column generally designated by identifier 40, which it to the left of dashed line 37, comprises the talus 26, the navicular 22, the cuneiforms 18 and rays one, two and three of the metatarsals and of the phalanges. This corresponds to the inner section of the foot including the first three toes.

There are four arches of the foot. The “medial longitudinal arch” includes the calcaneus, talus, navicular, the lateral, middle and medial cuneiforms, and the first three metatarsals. In an ideal foot, the medial longitudinal arch is the highest of the three arches. The “lateral longitudinal arch” includes the calcaneus, cuboid, and the fourth and fifth metatarsals. The lateral longitudinal arch is typically lower and flatter than the medial arch. The two transverse arches are the “transverse tarsal arch” (comprising the cuneiforms, the cuboid and the base of the five metatarsals) and the “transverse metatarsal arch” (comprising the 5 metatarsal heads).

As will be detailed below, the present apparatus when in contact with a plantar surface of a foot adjusts selected foot structures, e.g., bones, joints, soft tissues, preferably initially in the midfoot region and subsequently in regions surrounding the mid-foot region. Adjustment of foot structures in a mid-foot region initially places the foot in a restored or adjusted state, wherein the relationship between the foot bones is clinically optimal. As used herein, an “initial bone state” intends the relationships of the bones in a patient's foot in a first, initial or unrestored configuration/relationship before adjustment or manipulation of the bones, such as by treatment with an apparatus as described herein. A “restored bone state” refers to the configuration/relationship of foot bones that is different from an initial bone state, and in a preferred embodiment refers to the configuration/relationship of foot bones that approaches or is a physiologically or medically desired position, for example, for optimal joint congruency and function. The apparatus includes structures for constructing an image or ‘digital orthotic profile’ that is used in construction of a foot orthotic device to maintain the foot bones in its restored bone state; that is with foot structures in a clinically optimal position. Neither the midtarsal joint nor the subtalar joint is a guiding or controlling structure, but instead these joints are merely responsive to the position of other foot structures, in particular structures in the mid-foot.

FIGS. 1C-1E illustrate what is intended by the term mid-foot region. FIG. 1C shows the 1^{st}, 2^{nd} and 5^{th} rays of foot 10, designed as 42, 43, and 44, respectively. The 1^{st} ray is along dashed line 42 and extends from the phlanges of the big toe, along the first metatarsal, bisects the lateral cunieform and the navicular. The 2^{nd} ray is along dashed line 43, and extends from the phlanges of the second toe, the second metatarsal, bisects the medial cunieform and the navicular. The 5^{th} ray is along dashed line 44 and extends along the fifth or little toe phlanges, the 5^{th} metatarsal, and the lateral edge of the cuboid bone. The mid-foot in a preferred embodiment is the region denoted 46 in FIG. 1D and bounded by the 1^{st} and 5^{th} rays, the base of the metatarsals and the midtarsal joint. The “localized” mid-foot region is the region denoted 48 in FIG. 1E and is bounded by the 2nd and 5^{th} rays, the base of the metatarsals and the midtarsal joint.

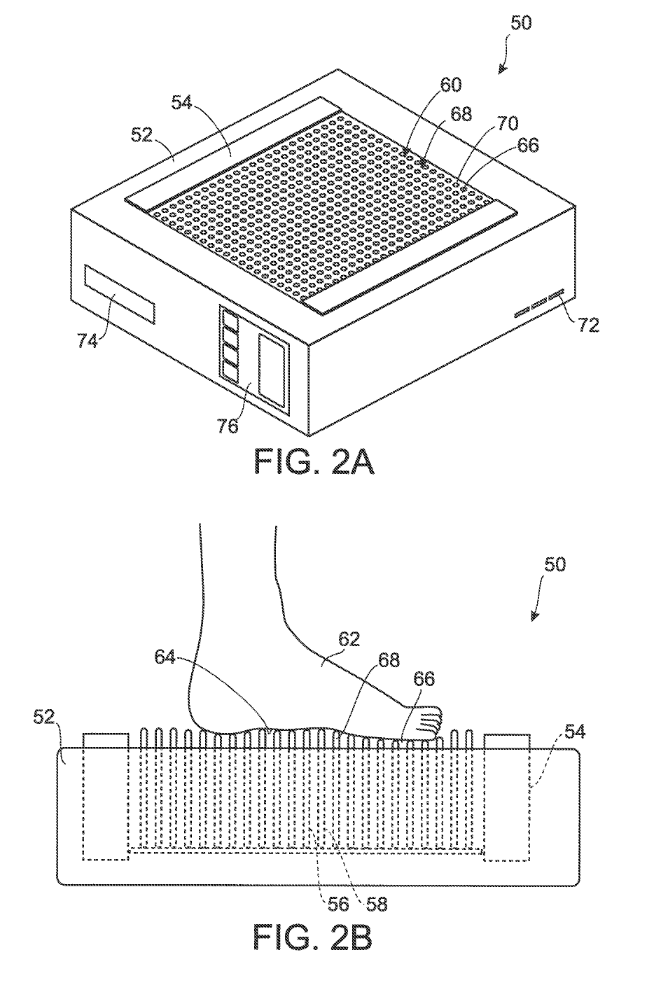

Turning now to the subject apparatus, a first embodiment is shown in FIGS. 2A-2B. Apparatus 50 is generally comprised of a housing 52 enclosing a support frame 54, the top portion of which is visible in FIGS. 2A-2B, and shown more fully in phantom in FIG. 2B. Disposed within support frame 52 is a plurality of movable pins or engagement structures, such as pins 56, 58 which are representative, to form a pin bed 60. Each pin is moveable between at least a first position and a second position, and is optionally movable to subsequent positions if desired. In one embodiment, a first pin position is a resting position in which the pin is withdrawn from contact with a foot 62 placed on the pin bed, and a second pin position is an engagement position in which the pin is in contact with the plantar surface 64 of the foot. In other embodiments, a first position is a resting position wherein a pin is in contact with a foot placed on the pin bed in such a way that little or no force is exerted by the pin against the plantar surface of the foot, and a second position is an engagement position in which the pin exerts a pressure or force on the plantar surface of the foot. An optional support plate 66 has a plurality of openings, such as openings 68, 70, through which a single pin can move as it travels betweens its resting position and its engagement position. The apparatus will generally, but optionally, include input and output ports, such as ports 72, 74, for connecting the apparatus to peripheral equipment, such as keyboards, computers, and other electronics. Appropriate on/off electronics 76 and display lights can optionally be included.

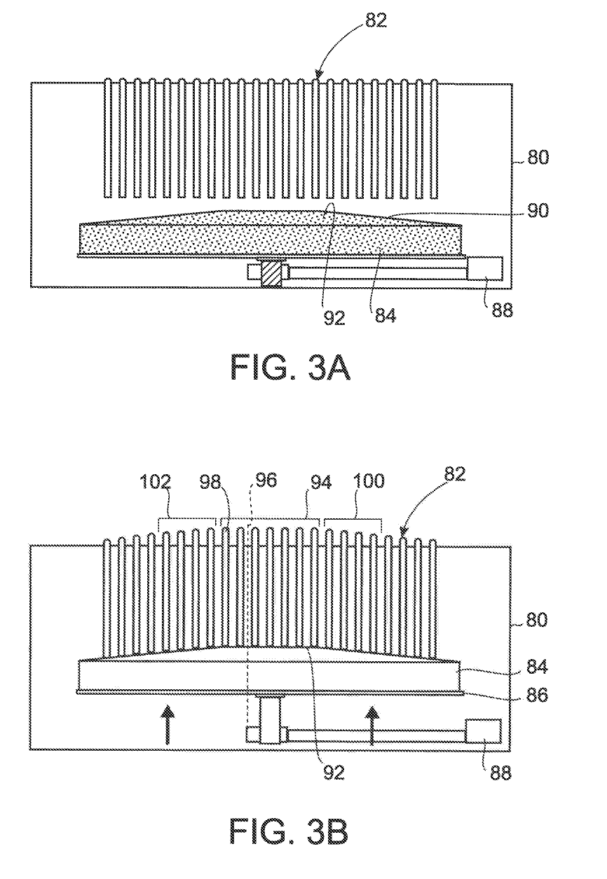

The apparatus also includes a biasing member, not shown in FIGS. 2A-2B, but shown and described with reference to FIGS. 3-9. As will be evident from the description of FIGS. 3-9, the apparatus can include one or more biasing members, and a variety of biasing members are contemplated, the embodiments shown herein merely exemplary of the concept. In a first embodiment, shown in FIGS. 3A-3B, a frame 80 provides structural support for an array of pins 82 and a biasing member 84. Biasing member 84 is positioned on a platform 86, visible in FIG. 3B, which is movable by a drive means 88.

Biasing member 84 has an upper surface 90 that has a preselected contour. The contour in the embodiment of FIGS. 3A-3B is such that an inner region 92 of the surface is a flat apex, relative to the upper surface 90. Regions of surface 90 surrounding the apex gently slope, giving the surface of the biasing member a pyramid-like contour. The biasing member is movable between a first position, as depicted in FIG. 3A, and a second position, as depicted in FIG. 3B. The biasing member in its first position is positioned such that its upper surface is not in contact with pins in the array or, alternatively, touches the proximal ends of selected pins correspondingly disposed above the flat apex region. As the biasing member travels from its first position to its second position, pins in the array of pins are engaged and displaced, in a pattern dictated by the preselected contour of the upper surface of the biasing member. In this embodiment, a first set of pins, designated by the bracket 94, is first contacted by the flat apex region of the biasing member. First set of pins 94 moves along their respective longitudinal axes, such as axis 96 of pin 98. Continued upward (with respect to the drawing) movement of the biasing member brings contact between the regions of the biasing member surface surrounding the apex and a second set of pins, designated by brackets 100, 102. Pins in the second set, once in engaging contact with the biasing member, move along their longitudinal axes. It is appreciated that as the biasing member continues movement, third, forth, and additional sets of pins come into contact with the biasing member and are urged into motion, for contact with a plantar surface of a foot on the array of pins. It is also appreciated that second and subsequent sets of pins are peripheral to the first set of pins, and preferably peripheral annularly. The number of pins in a set will vary, as can be appreciated, and can range from 1 pin to n−1 pins, where n is the total number of pins in the pin bed. More generally, the number of pins in a set will range from 3-10% of n, from 5-20% of n, from 8-25% of n, and from 10-30% of n, or in other embodiments, from 3-30% of n. As will be illustrated below, the number of pins in a set is not critical to the invention so long as the movement of the pin sets encounters and interacts with a foot to achieve movement of a foot structure from a first initial bone state to a second restored bone state. In preferred embodiments, a foot structure in the mid-foot region of the foot is adjusted to a restored bone state, and in more preferred embodiments, a foot structure in the mid-foot region of the foot is adjusted to a restored bone state prior to adjustment of a foot structure in a non-mid-foot region.

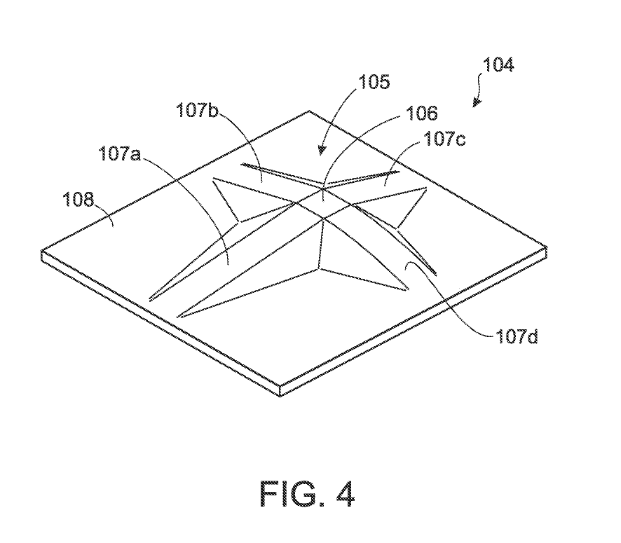

A skilled artisan will appreciate that the contour of the biasing member determines the initiation of movement of each pin in the array of pins, and the final position of each pin in the array. A variety of geometric shapes and surface contours of the biasing member are envisioned and contemplated. The pyramid-like shape is merely exemplary, and an alternative shape or surface contour is a terrace shape. Another exemplary shape is shown in FIG. 4, where member 104 has a surface contour that includes a raised structure 105. Structure 105 has an apex 106 that will engage one or more movable pins in a first set of an array of pins prior to engagement of pins peripheral to the first set. The apex of the biasing member is positioned within the apparatus such that a foot placed on the pin bed array is initially engaged by the first set of pins, i.e., the pins engaged by the apex 106 of structure 105, in a mid-foot region. Raised structure 105 has one or more slopes, such as slopes 107a, 107b, 107c, and 107d, that extend from the base surface 108 of biasing member 104 to apex 106. A skilled artisan will appreciate the possible variations in the actual dimensions of each slope, and the sidewalls between each slope.

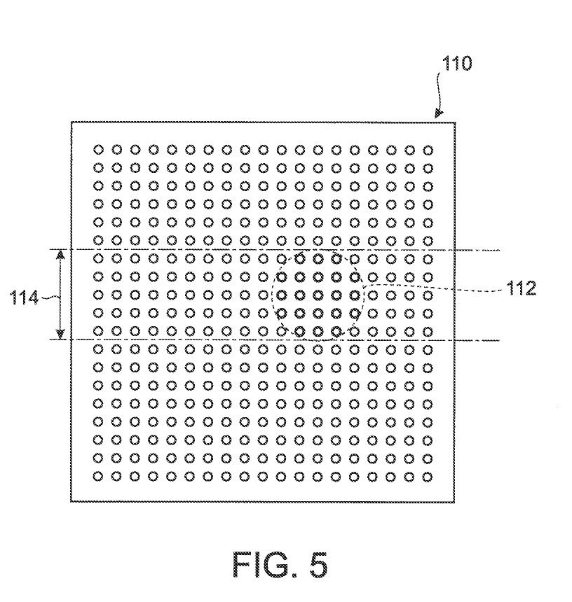

Generally, and in a preferred embodiment, the surface contour of the biasing member causes initial movement of a first set of pins in an inner region of the pin array. To illustrate, FIG. 5 depicts a top view of an array of pins 110. A first set of pins in an inner or center region of the array is identified by the pins within the dashed line 112. The first set of pins in the center region 112 is not necessarily in the mathematical center of the array, but will typically have surrounding pins on two, three, or four ‘sides’ of the region. More generally, the center region of the pin array can be considered all or a portion of a center band of pins, such as band 114 in FIG. 5. The center band has pins peripheral thereto, including both proximal and distal thereto, the peripheral pins forming second and subsequent sets of pins which are urged by one or more biasing members subsequent to the first set of pins. In use, as will be illustrated below, a foot is positioned such that all or a portion of the mid-foot is placed for contact with a first set of pins, such that structures in the mid-foot are contacted with pins before structures in the forefoot or rear foot.

In another embodiment, movement of first and subsequent sets of pins is achieved with a plurality of biasing members, rather than by a single biasing member as in FIGS. 3A-3B. In this embodiment, a first independently controlled biasing member is moved from its initial position to a second position, to urge pins in a first set. Second and optionally subsequent biasing members, preferably each independently controllable, are then moved from initial to second positions to urge second and optionally subsequent sets of pins. Illustrative examples of this embodiment are now provided.

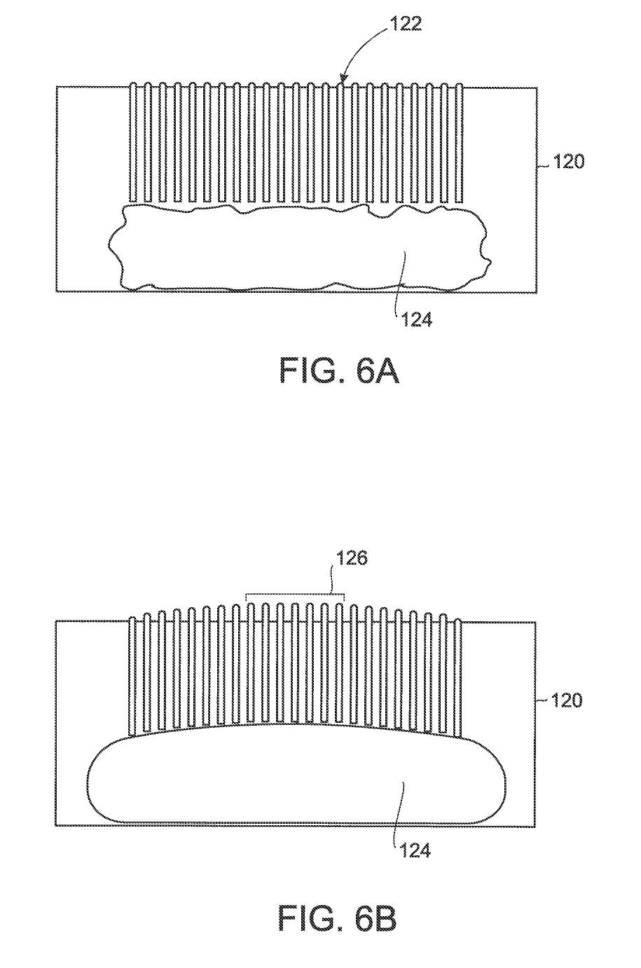

FIGS. 6A-6B illustrate another embodiment of a biasing member contemplated for use with the subject apparatus. A frame 120 provides structural support for a pin bed 122, and a biasing member 124. Biasing member is moveable incrementally from an initial position, as depicted in FIG. 6A, and a final position, such as that depicted in FIG. 6B. In this embodiment, biasing member is an inflatable structure, comprised of two or more materials that respond differently to the inflation pressure, or comprised of two or more distinct inflation structures that are inflated sequentially or separately. In this way, the biasing member is activated to urge a first set of pins, such as the pins designated by bracket 126, upward (with respect to the drawing) along their longitudinal axes. Continued inflation of the biasing member results in pressure applied to additional pins, resulting in movement of pins in addition to those pins in the first set.

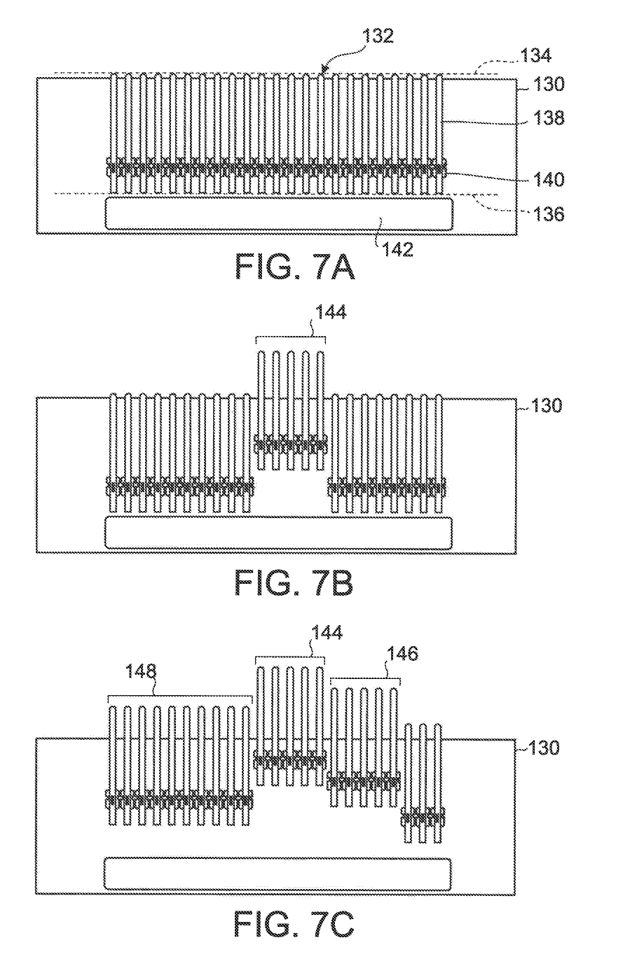

Another embodiment of a biasing member is shown in FIGS. 7A-7C. Here, a plurality of biasing members is provided within an optional support frame 130. A plurality of engagement structures 132 form an upper surface, designated by dashed line 134, and a lower surface, designated by dashed line 136. Each engagement structure in the plurality, such as engagement structure 138, has a dedicated biasing member, such as member 140 on engagement structure 138. The dedicated biasing members can be, for example, a piston driven by a pressure source, a servo-controlled motor adapted to raise an associated pin by a preselected distance, a spring (e.g., a constant force spring or a linear spring), a hydraulic, pneumatic or magnetic device, or the like. The necessary electronics to signal each biasing member is positioned, for example, in a structure 142. Each engagement structure in the plurality is moveable independently or in combination with other engagement structures in the plurality by a signal, such as an electric signal or pressure, communicated to the biasing member on the engagement structure. Upon receipt of a signal, one or more engagement structures are moved from their initial position, such as that depicted in FIG. 7A, to a second or subsequent position, such as that depicted in FIG. 7B, where a set of engagement structures 144 in a center region of the plurality has moved. Subsequent signals to other engagement structures causes movement of second engagement structure sets, such as set 146 in FIG. 7C, and third engagement structure sets, such as set 148 in FIG. 7C.

In one embodiment, all of the engagement structures in the plurality are moved simultaneously. In this embodiment, all or a portion of the engagement structures are different in length from each other or from another portion of engagement structures in the plurality so that as the engagement structures are moved simultaneously, from an initial flat surface, the resulting shape of the top surface of the engagement structures is of a desired shape or pattern. A skilled artisan can appreciate that the ability to control each engagement structure provides a vast number of possible preselected patterns of engagement structure movement. Under control of a computer, the position of each engagement structure can be adjusted as desired, and the engagement structures forming any given set can be readily varied.

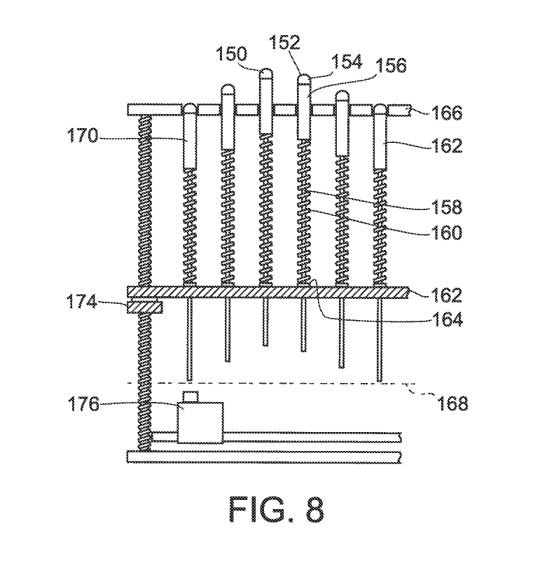

Another exemplary embodiment for biasing pins, also referred to herein as engagement structures, individually is depicted in FIG. 8 wherein differential, independent movement of pins is achieved by a spring associated with each pin. In this illustration, several pins in a plurality of pins, or engagement structures, are shown, and pins 150, 152 are representative. Each pin in the plurality comprises a distal tip with a sleeve, such as tip 154 and sleeve 156 on pin 152. The shaft of each pin, such as shaft 158 of pin 152, is enclosed by a spring, such as spring 160. Each pin is secured in a bottom plate 162 by a sealing structure, such as structure 164 on the shaft of pin 152. A top plate 166 has a series of openings aligned with each pin in the array and dimensioned for passage of each pin during its movement from an initial position to an engagement position. Dashed line 168 indicates the initial position of the proximal base of each pin in the array, and as seen the outer peripheral pins are in their initial positions and the center pins 150, 152 are moving toward or are in an engagement position due to upward (with respect to the drawing) travel of bottom plate 162. Differential travel of pins in the array is achieved through selection of the spring on each pin. For example, peripheral pins 170, 172 may have a shorter spring than neighboring pins, or a spring with a higher force constant than a neighboring pin, so that movement of force plate causes travel of pins with longer springs or lower force constants prior to causing movement of pins 170, 172. In this way, pins that will initially engage the mid-foot of a patient's foot placed on the plurality of pins, for adjustment of mid-foot structures prior to contact between the foot plantar surface and pins peripheral to the pins making initial contact in the mid-foot. The apparatus in FIG. 8 additionally includes one or more sensors, such as a force sensor 174 and/or a sensor 176 to determine the position of each pin at any time during operation of the apparatus, and in particular to determine the position of each pin in its engagement position.

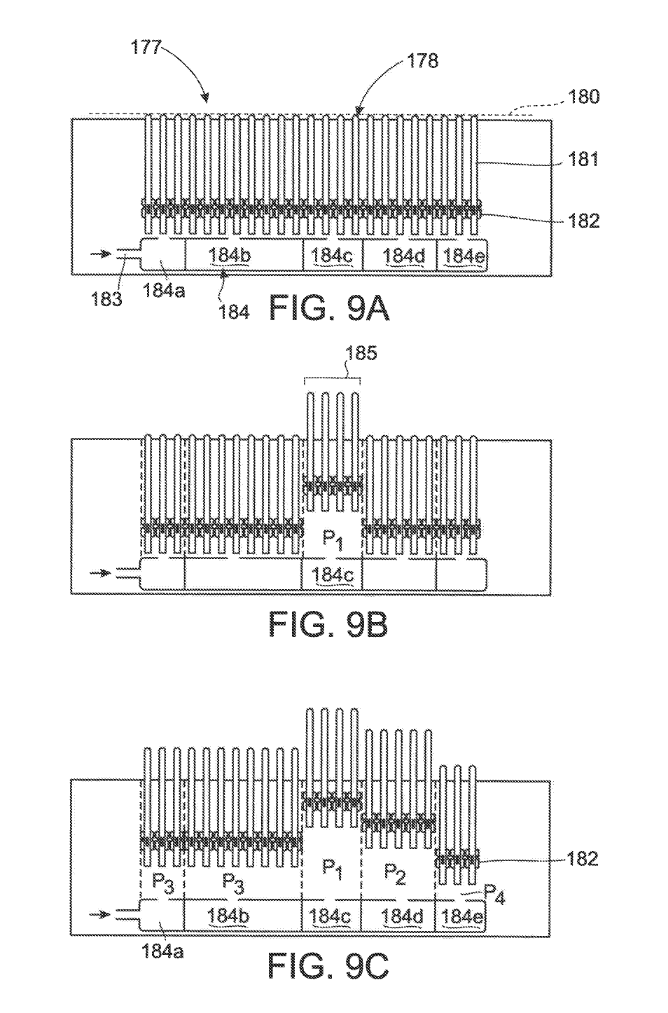

Another embodiment of the apparatus is illustrated in FIGS. 9A-9C. In this embodiment, an apparatus 177 comprises a plurality of engagement structures 178 that collectively define an upper surface, designated by dashed line 180. Each engagement structure in the plurality, such as engagement structure 181, has a dedicated sensor, such as Hall sensor 182 on engagement structure 181, to determine the position of the engagement structure at any time during operation of the apparatus. The apparatus also includes a manifold 184 with an inlet 183. Manifold 184 is partitioned into one or more zones, such as zones 184a, 184b, 184c, 184d and 184e, which are representative. Movement of a set of engagement structures, such as set 185, is effected by a change in pressure in a corresponding zone, such as zone 184c for set 185. A fluid, preferably a gas and more preferably an inert gas such as air or nitrogen, is introduced into manifold 184 via inlet 183. While not shown in detail in the drawing, appropriate valves, tubes, partitions and/or bladders are present in the manifold and/or the apparatus such that the gas can be directed into one or more desired zone(s) at the same or different pressure. By way of example, and with reference to FIG. 9B, a gas is introduced and routed to zone 184c to effect movement of engagement structures in the set of engagement structures 185. Gas in zone 184c is supplied to a selected pressure, P1, to urge pin set 185 into contact with a foot placed on surface 180. As discussed above, pin set 185 preferably contacts a mid-foot region of the foot, to adjust a bone in the mid-foot region to a restored bone state. Next, and with reference to FIG. 9C, gas is introduced into a second zone of the manifold, such as zone 184d, to achieve a selected pressure P2, which can be the same as or different from P1. Thereafter, gas is introduced into other zones, such as zones 184a, 184b and 184e, at the same or different pressures, indicated as P3 and P4 in FIG. 9C, to urge sets of pins controlled by the pressure in a corresponding zone into contact with a foot placed on the pin bed array. As can be appreciated, in this embodiment the biasing member of the subject apparatus is a gas, and more preferably, a pressurized gas.

The number of pins in a set can vary, as discussed above. The pressure applied to a pin or set of pins is also easily varied, as a skilled artisan will appreciate. In one embodiment, the pin bed array has at least two, preferably three, four, five, six, seven, eight, nine, ten or more separate regions that can be pressurized independently. In one embodiment, at least one zone is pressurized to a pressure greater than about 25 psig (1.7×10⁵ Pa), preferably greater than 30 psig (2.1×10⁵ Pa). In another embodiment, engagement structures in a set are urged by a biasing member that is a gas pressurized to a pressure between 5-40 psig (3.4×10⁴⁻2.8×10⁵ Pa). In another embodiment, a first pin set in the array is urged into an engagement position for contact with a foot at a first force sufficient to displace a bone in the foot, and second and optionally subsequent sets of pins are moved into an engagement position for contact with a foot at a second force that is less than the first force. In another embodiment, more than one pressure is applied to urge more than one set of pins into an engagement position for contact with a foot.

With reference again to FIG. 9A, in one embodiment an expandable material covers the surface 180, so that a foot placed on the surface is in direct contact with the expandable material. In another embodiment, each pin in the pin bed array is movably disposed in a cylinder in fluid communication with a manifold, and the amount of gas to be introduced into each cylinder is independently controllable. Alternatively, a set of n pins is movably positioned in a cylinder in fluid communication with a manifold, and the amount of gas can be controllably introduced or removed from the shaft to control movement of the set of n pins (where n is as defined above). In this way, the pressure in each cylinder can be varied to vary the pressure that urges each pin set into contact with a foot, and if desired after adjusting a foot to its restored bone state, the pressure in any individual cylinder or across all cylinders can be equalized.

With reference to FIG. 9C, when the pins or sets of pins are in a final desired engagement position, one or more sensors, such as Hall sensor 182, provide positional information for each pin in the array. The positional information is relayed to a computer, to construct a digital image of the foot, for construction of a foot orthotic that places one or more foot bones in a restored bone state.

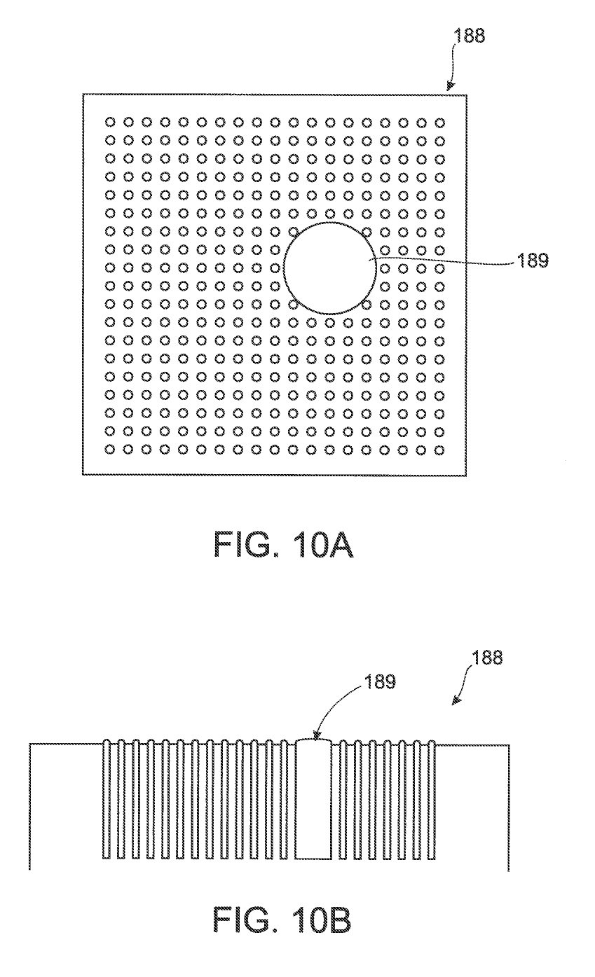

The exemplary devices described above each include an array of engagement structures that interact with one or more biasing members to achieve initial contact of a subset of pins in the pin bed array with a mid-foot region of a patient's foot. FIGS. 10A-10B illustrate another embodiment of an apparatus that achieves this desired sequence of contact. In this embodiment, an array of engagement structures 188 is dimensioned for receipt of a plantar surface of a foot. The array includes a center post member 189 that is positioned such that when a foot is placed on the array, post member 189 is in or will be in contact with a localized mid-foot region of the foot's plantar surface. Post member 189 can be movable in a longitudinal direction by a biasing member (not shown) or, in another embodiment, can be fixed and immovable. The post member is positioned in the array and dimensioned to contact the mid-foot region prior to contact between engagement structures peripheral to the post member and the foot's plantar surface. In this way, structures in the mid-foot are manipulated into an adjusted or restored state before structure peripheral to the mid-foot region are contacted by engagement structures in the array.

From the illustrative embodiments above, it can be appreciated that a skilled engineer can envision a variety of approaches to design an apparatus wherein one or more pins engage a foot at a desired position with a force sufficient to displace a structure (preferably a bone, ligament, connective tissue etc.) in the foot to manipulate the foot into a restored bone state. These variety of approaches include, in addition to those described herein, an array of pins wherein each pin in the array is raised simultaneously with the other pins in the array but sets of pins in the array contact the foot with different force (pressure). A higher force could, for example, be applied to the set of pins that contact the mid-foot region to adjust bones in the mid-foot region. In another variation, an array of pins is provided wherein the pins respond differently to an applied pressure to achieve differential application of pressure to a foot responsive to a commonly applied pressure to the pin bed. In other variations, it is contemplated to provide an apparatus wherein an engagement structure(s) physically contacts a foot in a position to achieve a restored bone state, and the profile of the foot surface is obtained in a non-physical contact manner, such as with a laser, to obtain a digital image of the foot in its restored state via physical contact only at the point of physical manipulation.

Accordingly, in an embodiment, an apparatus and a method comprise engaging a center engagement structure against a localized mid-foot region of a plantar surface of a subject's foot to adjust one or more mid-foot bones into a restored bone state and determining a surface map of the plantar surface of the foot with the mid-foot bone in its restored bone state. The surface map of the foot surface can be determined using a sensor that is not in physical contact with the foot or an engagement structure. For example, a laser can be used as the sensor, where the physical structure of the laser sensor does not contact the foot or the engagement structure, although the light beam emitted from the laser will contact the foot or the engagement structure. It will also be appreciated that in another embodiment, one or more engagement structures, in addition to the central engagement structure(s), can contact the plantar surface, and the position of the one or more additional engagement structures determined from which a surface map or profile of the plantar surface of the foot in its restored state is obtained.

A skilled artisan will appreciate that an alternative embodiment of the apparatus comprises an array of pins positioned in the apparatus in a first position for engagement with a plantar surface of a foot. The pins move independently to a second position subsequent to engagement with the plantar surface. One or more biasing members control or resist movement of the pins from the first to second position, where the one or more biasing members control or resist such movement at different pressures. By way of example, the apparatus of FIG. 8 can be modified such that the pins are in an initial “raised” position for engagement with a foot. As the foot presses on the pins, the pins are displaced or “lowered” to a second position. A biasing member associated with each pin, e.g., in this embodiment a spring on each pin, resists the downward force applied to each pin, where the resistance can differ according to the force constant of each spring, or in embodiments where the biasing member is a fluid, the pressure of the fluid. Higher resistance to the applied force for pins in a first set in, for example, the midfoot region of the foot, relative to the resistance to the applied force for pins in a second set in a region other than the midfoot, can achieve adjustment of the foot to a restored bone state. Accordingly, the apparatus according to embodiments described herein comprise one or more biasing members configured to exert a force on one or more pins, or one or more sets of pins, in the plurality of pins. In one embodiment, the biasing member(s) exerts a force by resisting pressure applied to the pins and in another embodiment the biasing member(s) exert a force by urging one or more pins or pin sets from first to second positions.

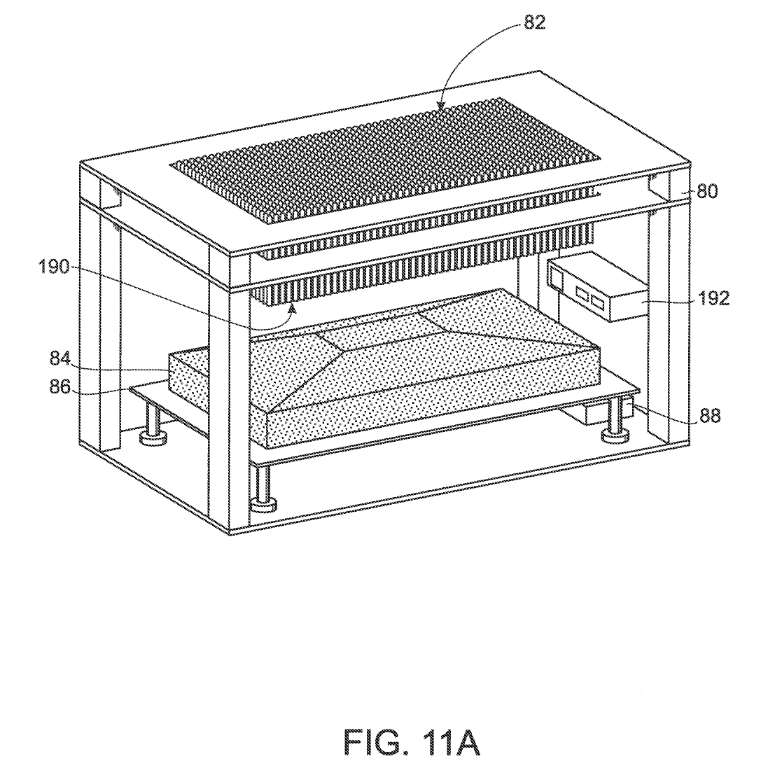

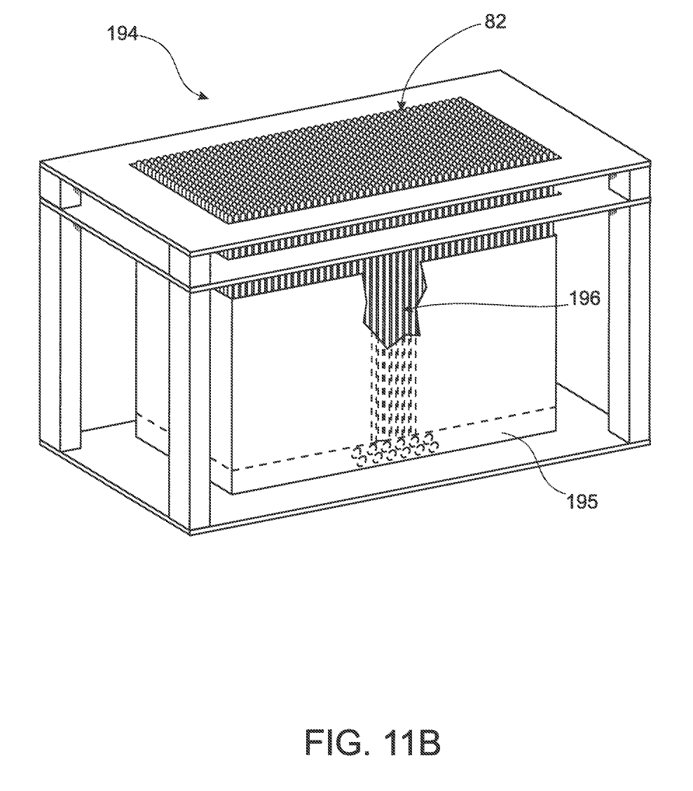

FIG. 11A provides a more detailed perspective view of the embodiment described in FIGS. 3A-3B, where the same structural features are identified by the previously assigned numerical identifier. Frame 80 supports a plurality of pins 82, each pin independently movable between a first position and one or more subsequent positions. A biasing member 84 is disposed within the frame, in contact with a movable platform 86. Movement of platform 86 is controlled by a driver 88. Biasing member 84 has a preselected contoured surface, shown here as a pyramid-like contour with an off-center flat apex. When urged upward, the contoured surface contacts a lower surface 190 of a set of pins that are correspondingly engaged by the flat apex of the biasing member. Continued upward movement of the biasing member engages additional pins, and/or sets of pins, in accord with the preselected contour of the biasing member.

The material of which the biasing member is manufactured is varied, and will depend on the embodiment. For biasing members as depicted in FIGS. 3A-3B, materials such as, but not limited to, rubbers, elastomers, plastics, and foams are contemplated. In one embodiment, the biasing member is made from a composite of two or more materials, such as a first and second materials with different densities, durometers, porosities, densities, and the like. In one embodiment, the biasing member is comprised of a composite foam comprised of a first material with a first durometer or a first density and a second material with a second durometer or a second density. Composites of viscoelastic foams are exemplary.

FIG. 11B is a perspective view of another embodiment of an apparatus 194. A pin bed array 82 is comprised of a plurality of independently movable pins. Each pin in the array is individually and independently responsive to one or more biasing members, which in this embodiment is a fluid, preferably pressurized fluid, preferably a gas, such as air or nitrogen. In exemplary apparatus 194, the pressurized fluid is supplied by a manifold 195, partially shown in phantom. The manifold comprises sufficient valves, tubing and connections to permit control of each pin individually and/or of pins in defined sets of pins. In one embodiment, each pin is individually controllable by a biasing member, and the pins can be grouped into sets for simultaneous movement of pins within a set. In one embodiment, and by way of example, a first set comprised of four pins, such as pin set 196, is urged from an initial resting position (as shown in FIG. 11B) to an engagement position (not shown in FIG. 11B), followed by a second set of eight pins urged from a resting position to an engagement position (not shown in FIG. 11B). In a preferred embodiment, the first set of pins is urged into its engagement position by introducing a gas into a housing or cylinder for each pin in the set, the cylinder in fluid communication with the manifold and the pin movable in a longitudinal direction fixed within a corresponding cylinder. The number of pins in each set can be different or can be the same, as discussed above. Each pin or each set of pins can be urged into its engagement position at a selected force applied by its corresponding biasing member. For example, the first set of pins can be urged by a first pressure P1 that is higher than a second pressure P2 that urges a second set of pins into their engagement positions.

With respect to all embodiments herein, the dimensions and density of the pins in the array of pins will vary. In one embodiment, each pin has an outer diameter of between about 0.0624 inches and about 0.250 inches, more preferably between about 0.08 inches and about 0.2 inches. The pin density, in one embodiment, is between about 6 pins/in² and about 12 pins/in², more preferably between about 8 pins/in² and about 12 pins/in², and still more preferably between 9-11 pins/in². The force produced by the pins when urged by the biasing member is typically on the order of about 0.02-5.0 lbf per pin, more preferably of between about 0.02-2.0 lbf per pin.

With reference again to FIG. 11A, in one embodiment, the apparatus further comprises a sensor 192 to determine a position of one or more pins in the array of pins. In one embodiment, a single sensor that determines the position of each pin in its final position is provided. In another embodiment, two or more sensors are provided. A variety of sensors are suitable for capturing positional information of each pin, such as lasers (including a one-dimensional laser, a two-dimensional laser, and a three-dimensional laser), an optical distance scanner, and the like. Photogrammetry sensing is also contemplated as a sensing means. A skilled artisan will appreciate that reflective surfaces, such as mirrors and metal-plated surfaces, can be positioned appropriately for reflection of laser beams. In one embodiment, each pin in the array of pins is associated with a magnet, and a Hall-effect sensor is associated with each pin. The array of Hall-effect sensors scans the array of pins to determine the position of each magnet associated with each pin. An exemplary arrangement of magnets associated with pins and an array of Hall-effect sensors is described in U.S. Pat. No. 5,640,779, which is incorporated by reference herein. Irrespective of the type of sensor selected, it will be appreciated that the sensor(s) is(are) operably connected to appropriate electronics to relay digital information about pin position, pin distance traveled, pin pressure or force, etc., for construction of a digital map of the pin bed array with each pin in its final position. This digital map, of course, represents a dimensionally correct image or map of a desired contour for a foot orthotic.

### III. Methods of Use

In another aspect, a method for determining a profile or contour for fabrication of a foot orthotic is provided. The method comprises engaging at least a center engagement structure against a mid-foot region or a localized mid-foot region of a plantar surface of a patient's foot to adjust one or more mid-foot bones into a restored state. One or more peripheral engagement structures is subsequently engaged against at least one annular region surrounding the mid-foot region to adjust the foot to a restored bone state or to adjust additional bones or tissue of the foot while maintaining the engagement of the center structure. Then, positional information of the engagement structures is obtained, and a surface map or orthotic profile based on the positional information is constructed.

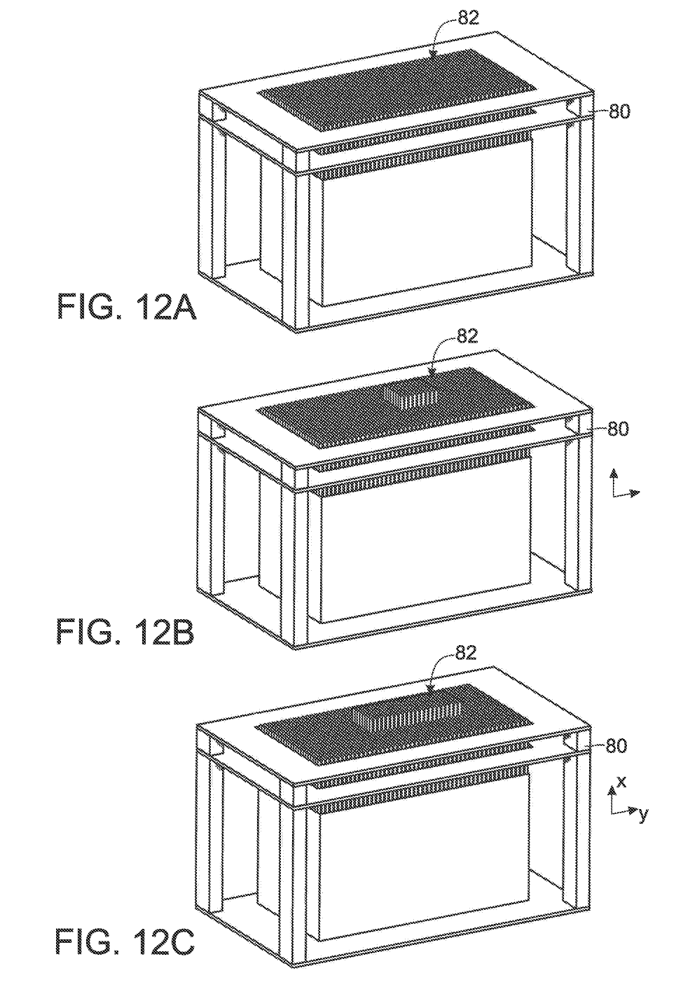

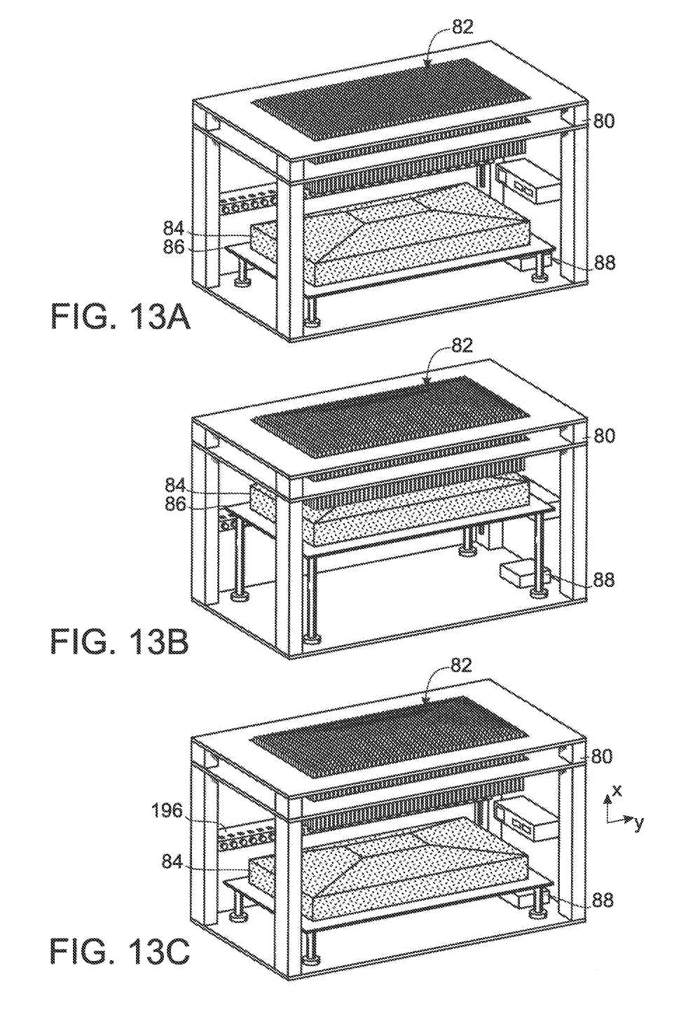

This method of using the apparatus described above to capture a profile (digital or physical) that informs a therapeutic, restorative contour for an individual foot will now be described with respect to FIGS. 12-13. With initial reference to FIGS. 12A-12C, where like structural elements from previous drawing figures retain previously assigned numerical identifiers, merely for the reader's convenience, a subject places a foot on an upper surface of a pin array 82. Although not shown in FIG. 12A, an optional flexible, expandable material can be used to cover the upper surface of the pin array. In a preferred embodiment, the subject is seated and the left or right foot is positioned on the pin array such that the calcaneus is at one end of a longitudinal center line of the pin array and the space between the 2^{nd} and 3^{rd} toes is at the other end of the center line. The subject is instructed to lean back in the chair to obtain an angle at the knee of between 90-110° and a straight line from the hip to the knee to the foot. If desired, a weight can be placed on the leg associated with the foot positioned on the pin array to fix the foot on the array. The system is then activated to initiate movement of pins in the plurality of pins. In one embodiment, a set of between 2-10 pins, preferably 2-6 pins, is urged by a biasing member in the form of a fluid pressurized to a first pressure P1, the set of pins urged from a resting position (FIG. 12A) into an engagement position (FIG. 12B) where the pins in the set exert a force on the subject's foot. Preferably, the first set of pins contacts the plantar surface of the foot in the mid-foot region, or the localized mid-foot region, and with a force sufficient to displace, adjust or move a bone in this region to achieve a restored bone state. In one embodiment, the first set of pins contacts the plantar surface to adjust one or more foot structures, but does not achieve a restored bone state. A second set of pins is urged from initial resting positions to engagement positions independently from the first set of pins, where independently from intends the second set of pins are urged into their engagement positions at the same time as pins in the first set but at a different pressure or force than the first set of pins, or independently intends the pins in the second set of pins are moved into their engagement positions subsequent to movement of the first set of pins, at the same or different pressure as the first set of pins, and preferably at a second lower pressure. The first set of pins can remain in contact with the foot at the first pressure P1 or, alternatively, the pressure applied to the first set of pins can be adjusted to P2. In one embodiment P2 is less than P1. In one embodiment, the first set of pins and second set of pins when engaged with the foot plantar surface adjust a foot structure to achieve a restored bone state.

An optional third set of pins can be urged at a third time or at a third pressure P3 from their resting positions to engagement positions. The first and second sets of pins can remain at P1 and P2, respectively, or can remain at P2, or can be adjusted to P3 so that the pressure for all raised pins is equilibrated. In a preferred embodiment, P3 is less than P2 which is less than P1. Optionally, fourth and subsequent sets of pins can be moved from resting to engagement positions at fourth and subsequent times or pressures applied by biasing members (e.g., pressurized fluid, e.g, pressurized gas), as can be appreciated.

The pattern in which the pin sets are raised and/or the pressure applied to each pin set will and can vary according to the subject's characteristics (weight, height, body mass index), foot anatomy and/or any particular orthopedic need of the subject. In one embodiment, an apparatus with at least six pin sets, preferably at least eight pin sets, and more preferably at least 10 pin sets is provided, wherein each set of pins is urged into contact with a plantar surface at a pressure different from another pin set. In another embodiment, the pressure or force applied to a first pin set is different from the pressure or force applied to a subsequent pin set to initiate movement of pins in the set to an engagement position, and thereafter the force or pressure across the pin sets is equalized. After movement of all pin sets in the array or after adjustment to the bones in the foot is complete, the position of each pin is ascertained by a means described herein, to obtain a digital profile for an orthotic that achieves a restored bone state for the subject.

Turning now to FIGS. 13A-13C, the biasing member embodiment of FIGS. 3A-3B is used for further illustration of the methodology, but it will be appreciated that the method applies to any of the embodiments described herein or discernable to a skilled artisan based on the description herein. Like structural elements retain previously assigned numerical identifiers, merely for the reader's convenience.

Use of the apparatus of this embodiment is initiated by placing a foot on the upper, exposed surface of pin bed 82. The patient can be standing or seated when the foot is placed on the bed. The pins are in an initial, resting position, as depicted in FIG. 13A. Movement of the biasing member (or members) is initiated by the driver or controller, such as driver 88. As the biasing member moves, the apex region of the biasing member contact a first set of pins in the pin bed, displacing the pins from their initial resting position to a second position. The first set of pins engages the mid-foot region of the foot. Continued movement of the biasing member, as shown in FIG. 13B, results in contact of the biasing member's upper surface with additional pins and sets of pins, which engage the foot plantar surface. As can be appreciated, continued movement of the biasing member to engage second and subsequent sets of pins causes continued pressure on the first set of pins, causing the first set of pins to probe deeply into the soft tissue in the mid-foot region, and adjust one or more bones in this region. Preferably, the first set of pins engages the mid-foot with sufficient force to adjust one or more bones therein prior to sufficient engagement of a second set of pins to adjust foot structures not within the mid-foot. That is, clinically, it is desirable to cause adjustment of the bones in the mid-foot prior to substantial contact of a second set of pins with the foot. In this way, the clinically therapeutic adjustment to the mid-foot is achieved, and the foot plantar surface responds to this adjustment, which is captured when the second or subsequent sets of pins contact the foot surface.

Once the pins are in a final position, they can be locked or secured in place by a suitable mechanism in the apparatus (not shown in FIGS. 13A-13C). The patient can remove his/her foot from the pin bed, if desired. Then, the position of each pin is determined using a sensor, and FIG. 13C a non-contact type of sensor is illustrated, and more specifically an optical sensor assembly sensor 198 is used. The sensor travels in the “y” direction, to scan each pin in the array to determine positional information. The information is transferred digitally to a computer, connected to the apparatus via suitable ports (FIG. 1). The position of each pin in the plurality defines a surface map that is a dimensionally correct image or map of a contour for a therapeutic foot orthotic for that foot.

It will be appreciated that use of the apparatus as depicted in FIGS. 13A-13C is exemplary, and that modifications to the apparatus and the sequence of events in use are contemplated. For example, in the embodiment of the apparatus wherein pins in a pin bed array are in a first position for engagement with a foot plantar surface, and a users places his/her foot on the pin bed array, one or more biasing members are provided to exert a force on one or more pins in the array to resist the force applied by the user. This embodiment as well as the embodiment discussed above wherein a biasing member moves to urge pins from first to second positions both comprise the feature that at least one biasing member is configured to exert a force on one or more pins in the array. In embodiments where the at least one biasing member comprises two or more biasing members, the force exerted by the biasing members can be the same or different, and are preferably different so that the force applied to selected regions of the foot differ.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. An apparatus, comprising:
a plurality of pins, each pin independently movable along a longitudinal axis, and
one or more biasing members configured to exert a force on one or more pins in said plurality of pins, such that a first set of pins in said plurality of pins is moved from a first position to a second position independent of a second set of pins in said plurality of pins.

2. The apparatus of claim 1, wherein pins in the first set of pins are moved prior to movement of pins in the second set of pins.

3. The apparatus of claim 1, wherein pins in the first set of pins are moved by a first force applied by a biasing member in the one or more biasing members to the pins in the first set of pins, the first force different from a second force applied by a biasing member in the one or more biasing members to pins in the second set of pins.

4. The apparatus of claim 1, wherein the one or more biasing members comprise at least two biasing members.

5. The apparatus of claim 4, wherein a first biasing member is dedicated to achieve movement of the first set of pins and a second biasing member is dedicated to achieve movement of the second set of pins.

6. The apparatus of claim 1, wherein said one or more biasing members comprise a plurality of biasing members.

7. The apparatus of claim 6, wherein each biasing member is dedicated for achieving movement of a single pin in the plurality of pins.

8. The apparatus of any one of claims 1-7, wherein the one or more biasing members are comprised of a pressurized fluid.

9. The apparatus of claim 8, wherein a first biasing member exerts a first pressure on pins in the first set of pins to move the pins in the first set of pins along their longitudinal axes, and a second biasing member exerts a second pressure on pins in the second set of pins to move the pins in the first set of pins along their longitudinal axes, where the first pressure is different that the second pressure.

10. The apparatus of claim 9, wherein the first pressure is higher than the second pressure.

11. The apparatus of claim 1, wherein said one or more biasing members is configured for contact with a third set of pins in said plurality of pins such that pins in the third set move along their longitudinal axis independent from movement of pins in said first set of pins or said second set of pins.

12. The apparatus of claim 11, wherein the third set of pins are subsequent to movement of pins in the first set of pins or wherein the third set of pins are moved by a pressure applied to the third set of pins that is different from a pressure applied to the first set of pins.

13. The apparatus of claim 1, wherein said first set of pins is within a center region of the plurality of pins and said second set of pins surround the periphery of the center region.

14. The apparatus of claim 1, wherein the plurality of pins collectively define an upper surface and a lower surface, and wherein said one or more biasing members is a single biasing member movably secured within the support frame.

15. The apparatus of claim 1, further comprising a sensor to determine a position of one or more pins within the plurality of pins.

16. A method for obtaining a restored bone state in a foot, comprising:
placing a plantar surface of a foot on an apparatus according to claim 1, the plantar surface placed on an upper surface defined by the plurality of pins, wherein said plurality of pins are in an initial position;
causing movement of pins in the plurality of pins via the one or more biasing members, such that at least a first set of pins adjusts one or more bones in the foot to a restored bone state and a second set of pins additionally engages the foot plantar surface; and
determining a position of each pin in at least the first pin set and the second pin set to obtain a profile of the foot in its restored bone state.

17. The method of claim 16, wherein causing comprises causing the first set of pins and the second set of pins to engage the foot plantar surface to adjust one or more bones to a restored bone state.

18. The method of claim 16, wherein causing comprises causing the first set of pins to engage the foot plantar surface to adjust one or more bones to a restored bone state prior to engaging the second set of pins with the foot plantar surface.

19. The method of claim 16, wherein determining comprises determining by means of a sensor a position of each pin.

20. The method of claim 17, wherein the method further comprises transferring positional information of each pin to a computer.

21. The method of claim 16, wherein said determining comprises determining a position of each pin in at least the first pin set and the second pin set to obtain a profile for construction of a foot orthotic or a series of foot orthotics to be worn sequentially.

22. The method of claim 16, wherein said causing further comprises causing the first set of pins to adjust one or more bones in the mid-foot region of the foot, and causing a third set of pins in the plurality of pins to move via the one or more biasing members, such that pins in the third set of pins engage the foot plantar surface at a region other than the mid-foot region.

23. A method, comprising:
placing a plantar surface of a foot an apparatus comprising (i) a plurality of pins, wherein each pin in the plurality of pins is independently movable along a longitudinal axis, and (ii) one or more biasing members configured for contact with one or more pins in said plurality of pins, such that a first set of pins in said plurality of pins is moved along the longitudinal axis of each pin in the first set independent of a second set of pins in said plurality of pins;
causing movement of the one or more biasing members such that at least some of the pins in the plurality adjust one or more bones in the foot to an adjusted position; and
determining a position of each pin in at least the first pin set and the second pin set.

24. The method of claim 23, wherein causing comprises causing movement such that the first set of pins adjusts one or more mid-foot bones to an adjusted position.

25. The method of claim 23, wherein the one or more biasing members is a fluid, and causing comprises causing movement of the one or more biasing members by pressurizing the fluid.

26. The apparatus of claim 25, wherein causing comprises causing movement of a first biasing member at a first pressure, to achieve movement of the first pin set, and movement of a second biasing member at a second pressure, to achieve movement of the second pin set.

27. The method of claim 23, wherein determining comprises determining the position of an end of each pin in at least the first pin set and the second pin set that engages the foot plantar surface to obtain a positional point of each pin, said positional points collectively defining a surface map.

28. A method, comprising:
engaging a center engagement structure against a localized mid-foot region of a plantar surface of a subject's foot to adjust one or more mid-foot bones into a restored bone state;
determining a surface map of the plantar surface of the foot with the mid-foot bone in its restored bone state.

29. The method of claim 28, wherein determining comprises determining a surface map of the plantar surface using a sensor which does not physically contact the foot.

30. The method of claim 29, wherein the sensor is a laser.

31. The method of claim 28, further comprising engaging one or more peripheral engagement structures against a region other than the mid-foot region to contact the plantar surface while maintaining the engagement of the center structure.

32. The method of claim 31, further comprising obtaining positional information of the one or more peripheral engagement structures, and from the positional information determining the surface map.
